(19) United States
(12) Patent Application Publication
(10) Pub. No.: US 2011/0291425 A1
(43) Pub. Date: Dec. 1, 2011

(54) LOW CO2 EMISSIONS SYSTEMS

(76) Inventor: James Charles Juranitch, Ft. Lauderdale, FL (US)

(21) Appl. No.: 12/998,692

(22) PCT Filed: Nov. 19, 2009
(86) PCT No.: PCT/US09/06206

§ 371 Date: Aug. 15, 2011

Related U.S. Application Data
(60) Provisional application No. 61/199,760, filed on Nov. 19, 2008.
(60) Provisional application No. 61/199,761, filed on Nov. 19, 2008.
(60) Provisional application No. 61/199,828, filed on Nov. 19, 2008.
(60) Provisional application No. 61/199,837, filed on Nov. 19, 2008.
(60) Provisional application No. 61/201,464, filed on Dec. 10, 2008.
(60) Provisional application No. 61/208,483, filed on Feb. 24, 2009.
(60) Provisional application No. 61/270,035, filed on Jul. 3, 2009.

(30) Foreign Application Priority Data
Jul. 1, 2009 (US) PCT/US2009/003934

(51) Int. Cl. H02K 7/18; C07C 27/06; A01G 1/00; C01C 1/04

(52) U.S. Cl. 290/1 R; 423/352; 423/359; 518/704; 47/1.4

(57) ABSTRACT

Systems and methods of generating power or producing gaseous products generate CO₂ as a waste product or as a greenhouse gas. Rather than being discharged into the atmosphere, the CO₂ is employed in a bioreactor to enhance the growth of algae. The algae then becomes a commercial product, or it can be consumed as fuel in the generation of power or the production of a gaseous product.

### RELATIONSHIP TO OTHER APPLICATIONS

This application claims the benefit of the filing date of U.S. Provisional Patent Application Ser. No. 61/270,035, filed Jul. 3, 2009, Confirmation No. 9380 (Foreign Filing License Granted); and is a continuation-in-part of copending International Patent Application Ser. No. PCT/US2009/003934, filed Jul. 1, 2009, which claims the benefit of the filing date of U.S. Provisional Patent Application Ser. No. 61/133,596, filed Jul. 1, 2008; and is a continuation-in-part, and claims the benefit of the filing dates of, U.S. Provisional Patent Application Ser. Nos. 61/199,837, filed Nov. 19, 2008; 61/199,761 filed Nov. 19, 2008; 61/201,464, filed Dec. 10, 2008; 61/199,760, filed Nov. 19, 2008; 61/199,828 filed Nov. 19, 2008, and 61/208,483, filed Feb. 24, 2009; the disclosures of all of which are incorporated herein by reference.

### BACKGROUND OF THE INVENTION

1. Field of the Invention

This invention relates generally to systems for generating power and systems for producing gas products, and more particularly, to a system and method of employing exhaust and waste CO₂ to enhance the growth of algae as a commercial product and as a feedstock.

2. Description of the Related Art

Prior to the 1980's it was normal and considered good practice to design, build, and operate dedicated manufacturing plants for most every product that was produced on a large scale in the world. Shortly after that period many industries became enlightened to the merit of flexible machining, and flexible manufacturing. Much of this paradigm shift was accomplished on the back of the modern microprocessors. This device allowed more enlightened design, and more importantly complex control, and precise control of involved processes. Many new inventions were required in the wake of this manufacturing revolution. Instead of an assembly line in the past making only one dedicated product, current best in class assembly lines make up hundreds of different products and models, in batch sizes of one, with no perceived loss of productivity or additional set up time. This clever thought process, and design, has netted many benefits to industries like the automotive world, heavy duty engines, and other hard goods manufacturers.

The energy world has not yet embraced this concept. There is a need to produce energy products in a more efficient and cleaner process than any carbon based process presently in use. One implementation of this concept does not use fossil fuels. In another implementation, the use of fossil fuels (such as coal) is minimized where possible. This is essential in developed countries, such as the United States, that have an elevated need for a “clean coal” electricity generation system, and for the manufacture of other fossil fuel energy dependent products, such as plastics, gaseous fuels, and fertilizers. In the United States and other developed countries, there is an ongoing effort to reduce the dependence on imported oil. It is, therefore, an object of this invention to achieve these goals in an environmentally friendly way.

Current power generation plants have only one primary process; i.e., to produce electricity by burning fuel and consequently emitting pollutants, such as green house gasses. One of the significant disadvantages of conventional power plants is that when they are brought off-line, and then restarted, they are unacceptably inefficient and produce exceeding amounts of harmful emissions. Modern power plants do not efficiently enable the throttling back of production of electrical power, and therefore they are operated continuously near the designed load limit. Since electrical power cannot be stored, power plants are frequently shut down and restarted in response to the varying demand for electrical power by consumers on a day-to-day basis, and as a result of differences in demand between day and night conditions.

With few exceptions (e.g., hydroelectric, wind, and nuclear power generation systems) power plants burn huge amounts of fossil fuel at relatively low efficiencies. The average efficiency of coal power plants in the United States is approximately 34%. Natural gas plants are slightly more efficient.

The predominant green house gas produced by power plants is CO₂. In the present state of the art, coal plants in the United States produce on average approximately 2.01 Lbs of CO₂ per KWH of power. Natural gas and petroleum plants produce about 1.6 Lbs of CO₂ per KWH. These are “carbon positive” green house gasses, in that they were removed from the ground and released to the atmosphere. In a carbon neutral process no new emissions are released into the atmosphere. In other words, nothing that has been removed from the ground is released into the atmosphere. Only existing carbon and green house gasses that are already in circulation are processed and released.

In a carbon negative process green house gasses are removed on a net basis from the atmosphere into a captured state. An example would be to extract CO₂ from the atmosphere and capture it into a hard substance, such as a plastic. A better product would be fertilizer since it could then be used to grow plants and food that continue to capture CO₂.

It is, therefore, an object of this invention to provide a power generation system that reclaims heat energy that otherwise would be exhausted into the atmosphere.

It is another object of this invention to provide a power generation system that operates at higher efficiency than conventional power generation systems.

It is also an object of this invention to provide a power generation system that eliminates the need for load cycling in response to consumer demand for power.

It is yet another object of this invention to provide a power generation system that greatly reduces emissions of CO₂.

In addition to the foregoing, the current method of producing ammonia typically begins with fossil fuels such as coal, oil, natural gas, propane, butane, naphtha, etc. that are processed to liberate hydrogen. This known approach disadvantageously strains limited resources. The known processes liberate significant amounts of carbon dioxide and other green house gasses that are believed by some to contribute to global warming. In addition to environmental effects, the known processes have resulted in political unrest, such as in China where the population battled over the rationing of fertilizer containing ammonia. The political unrest resulted from the fact that the fossil fuels needed to produce the ammonia were preferentially redirected to other fuel starved areas.

In 2006 the worldwide production of ammonia was approximately 146.5 million tons. It is believed that political problems will worsen in the future. For example, it was estimated that in 2003 83% of all ammonia produced was used to produce fertilizer. Moreover, it has been published that over 33% of the worlds food supply is generated through the use of fertilizer, and some have argued that the percentage is higher. It is therefore evident that with reasonably population growth and increasing competition for arid land, the reliance on fertilizer will only increase.

In 2004 China was the largest producer of fertilizer for the world at 28.4%, followed by India at 8.6%, Russia at 8.4%, and the United States at 8.2%. None of the operations in these countries use large scale renewable resources. Europe, up until the end of WWII, used a 60 MW hydroelectric power plant at Vermork, Norway to produce ammonia. The plant produced the required key ingredient, hydrogen, using an electrolysis process. Electrolysis is generally not economically feasible for producers who are not blessed with hydroelectric power. At that time, much of the ammonia was used to produce munitions for the war, and the economics of such application of resources was not questioned. The foregoing notwithstanding, the Vermork site was a prominent example of ammonia production using a non-carbon-liberating base of production to date.

Plasma melters are now becoming a reliable technology that is used to destroy waste. At this time there are few operational plasma melter installations but the technology is gaining acceptance. It is a characteristic of plasma melters that they produce a low BTU syngas consisting of several different elements. If the plasma melters are operated in a pyrolysis mode of operation, they will generate large amounts of hydrogen and carbon monoxide. The syngas byproduct typically is used to run stationary power generators, and the resulting electric power is sold to the power grid.

It is, therefore, an object of this invention to provide a system for liberating hydrogen.

It is another object of this invention to provide a system for liberating hydrogen on a large scale and that does not require large electrical generation resources.

It is also an object of this invention to provide a system for liberating hydrogen that does not require consumption of natural resources.

It is a further object of this invention to provide a method and system of producing ammonia inexpensively.

It is additionally an object of this invention to provide an inexpensive method of using hydrogen to produce ammonia.

It is yet a further object of this invention to provide an inexpensive method of using a plasma melter to generate large amounts of hydrogen.

It is also another object of this invention to provide a method of generating hydrogen wherein waste carbon dioxide is obtained from a renewable energy source and therefore does not constitute an addition to the green house gas carbon base.

### SUMMARY OF THE INVENTION

The foregoing and other objects are achieved by this invention which provides a method of manufacturing ammonia on a large scale. In accordance with an ammonia-producing aspect of the invention, the method includes the steps of:

supplying a fuel material to a plasma melter;

supplying electrical energy to the plasma melter;

supplying steam to the plasma melter;

extracting a syngas from the plasma melter;

extracting H₂ and CO₂ from the syngas;

supplying at least a portion of the CO₂ extracted from the syngas to a bioreactor for enhancing the growth of algae; and

forming ammonia from the hydrogen produced in said step of extracting hydrogen;

wherein the algae forms at least a portion of the fuel material in said step of supplying the fuel material to the plasma melter.

In accordance with an ethylene-producing aspect of the invention, the method includes the steps of:

supplying a fuel material to a plasma melter;

supplying electrical energy to the plasma melter;

supplying steam to the plasma melter;

extracting a syngas from the plasma melter;

extracting H₂ and CO₂ from the syngas;

supplying at least a portion of the CO₂ extracted from the syngas to a bioreactor for enhancing the growth of algae; and

forming ethylene from the hydrogen produced in said step of extracting hydrogen;

wherein the algae forms at least a portion of the fuel material in said step of supplying the fuel material to the plasma melter.

In accordance with a methane-producing aspect of the invention, there are provided the steps of:

supplying a fuel material to a plasma melter;

supplying electrical energy to the plasma melter;

supplying steam to the plasma melter;

extracting a syngas from the plasma melter;

extracting H₂ and CO₂ from the syngas;

supplying at least a portion of the CO₂ extracted from the syngas to a bioreactor for enhancing the growth of algae; and

forming methane from the hydrogen produced in said step of extracting hydrogen;

wherein the algae forms at least a portion of the fuel material in said step of supplying the fuel material to the plasma melter.

In accordance with a method of reclaiming carbon dioxide in an industrial process, there are provided the steps of:

obtaining an output gas from the industrial process;

delivering the output gas to a plasma melter;

delivering a fuel material to the plasma melter;

extracting CO and H₂ from the plasma melter;

converting the CO and H₂ into CO₂ and H₂;

delivering at least a portion of the CO₂ and H₂ to a reactor wherein the CO₂ and H₂ are converted to CH₄ and steam;

returning the CH₄ to the industrial process; and

delivering at least a portion of the CO₂ obtained in said step of converting the CO and H₂ into CO₂ and H₂ to a bioreactor for enhancing the growth of algae.

In one embodiment of the inventive method of reclaiming carbon dioxide in an industrial process, the output gas from the industrial process is CO₂. Prior to performing the step of delivering the output gas to a plasma melter there is provided, in one embodiment, the step of collecting the CO₂ from the industrial process. In a further embodiment, the output gas from the industrial process is an exhaust gas.

In accordance with a system aspect of the invention, there is provided a system for generating electrical power, the system including a reactor for producing a product gas in response to the consumption of a feedstock. A heat reclamation arrangement extracts heat from the product gas and forms heated steam. There is additionally provided a turbine having an input for receiving the heated steam, an outlet for exhausting spent steam, and a rotatory output. An electrical generator is coupled to the rotatory output of the turbine for producing electrical energy. Additionally, a bioreactor is in some embodiments of the invention arranged to receive CO₂ for enhancing the growth of algae.

In one embodiment of the invention, there is further provided the delivery of at least a portion of the algae as a fuel material to the plasma melter.

In accordance with a further method aspect of the invention, there is provided a method of operating an electrical power plant. In accordance with the invention, the method includes the steps of:

delivering a feedstock to a plasma melter to produce a product gas;

reclaiming heat from the product gas in a heat reclamation arrangement to form a super heated steam;

reclaiming CO₂ and H₂ from the product gas; and

delivering the CO₂ to a bioreactor for enhancing the growth of algae.

### BRIEF DESCRIPTION OF THE DRAWING

Comprehension of the invention is facilitated by reading the following detailed description, in conjunction with the annexed drawing, in which:

FIG. 1 is a simplified schematic representation of an embodiment of the invention wherein ammonia product is produced along with algae that is used as a fuel;

FIG. 2 is a simplified schematic representation of an embodiment of the invention wherein ethylene product and other carbon-based products are produced along with algae that is used as a fuel;

FIG. 3 is a simplified schematic representation of an embodiment of the invention wherein methane product is produced along with algae that is used as a fuel;

FIG. 4 is a simplified schematic representation of a still further specific illustrative embodiment of the invention, utilizing a Europlasma plasma melter and wherein methane product is produced along with algae that is used as a fuel; and

FIG. 5 is a simplified schematic representation of yet another embodiment of the invention showing a primary plant system, and wherein algae is produced that is used as a fuel.

### DETAILED DESCRIPTION

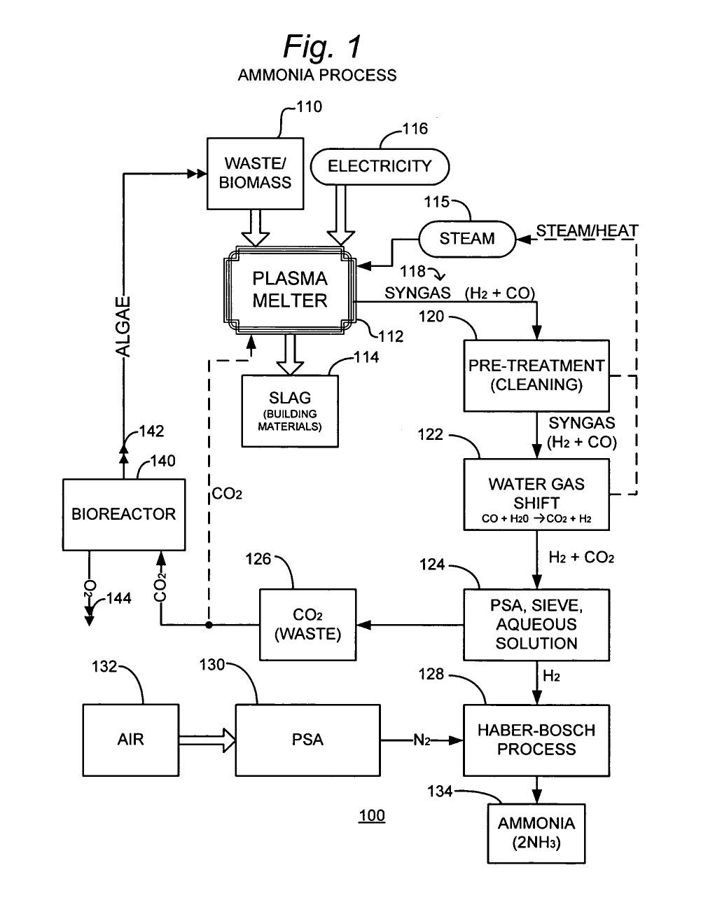

FIG. 1 is a simplified schematic representation of an embodiment of the invention wherein ammonia product is produced along with algae for use as a fuel. As shown in this figure, an ammonia producing system 100 receives municipal waste, or specifically grown biomass 110 that is deposited into a plasma melter 112. In the practice of some embodiments of the invention, the process is operated in a pyrolysis mode (i.e., lacking oxygen). Steam 115 is delivered to plasma melter 112 to facilitate production of hydrogen and plasma. Also, electrical power 116 is delivered to plasma melter 112. A hydrogen rich syngas 118 is produced at an output (not specifically designated) of plasma melter 112, as is a slag 114 that is subsequently removed.

In some applications of the invention, slag 114 is sold as building materials, and may take the form of mineral wool, reclaimed metals, and silicates, such as building materials. In some embodiments of the invention, the BTU content, plasma production, and slag production can also be “sweetened” by the addition of small amounts of coke or other additives (not shown), which in some embodiments of the invention includes fossil fuels. In other embodiments, the fossil fuels are combined to form a fossil fuel cocktail that includes, for example, a biomass material, municipal solid, waste and coal. In still other embodiments, the fossil fuels may be of a low quality, such as brown coal, tar sand, and shale oil.

The syngas is cooled, cleaned, and separated in a pretreatment step 120. The carbon monoxide is processed out of the cleaned syngas at the output of a Water Gas Shift reaction 122. The waste carbon dioxide 126 that is later stripped out is not considered an addition to the green house gas carbon base. This is due to the fact it is obtained in its entirety from a reclaimed and renewable source energy. In this embodiment of the invention, the energy source is predominantly municipal waste 110.

In some embodiments, the carbon dioxide is recycled into the plasma melter 112 and reprocessed into carbon monoxide and hydrogen, or carbon and O₂. A Pressure Swing Adsorption (PSA) process, molecular sieve, aqueous ethanolamine solutions, or other processes are used in process step 124 to separate out carbon dioxide 126. Hydrogen from process step 124 is delivered to a conventional Haber Bosch process 128, which is a well-known large scale high pressure process for producing ammonia, or other similar process, to produce ammonia 134. The required nitrogen is extracted from air 132 through a PSA 130 or any other conventional method. As previously noted, the hydrogen is, in some embodiments of the invention, extracted from the plasma melter. Pretreatment step 120 and Water Gas Shift reaction 122 generate heat that in some embodiments of the invention is used to supply steam to the plasma melter, or to a turbine generator (not shown), or any other process (not shown) that utilizes heat.

In accordance with a highly advantageous embodiment of the invention, the waste CO₂ 126 that is issued at process step 124 is delivered to a bioreactor 140 that produces algae at an output 142. The algae is produced using the waste CO₂ and is delivered as biomass 110 to plasma melter 112. In addition to the foregoing, bioreactor 140 generates O₂ at an output 144.

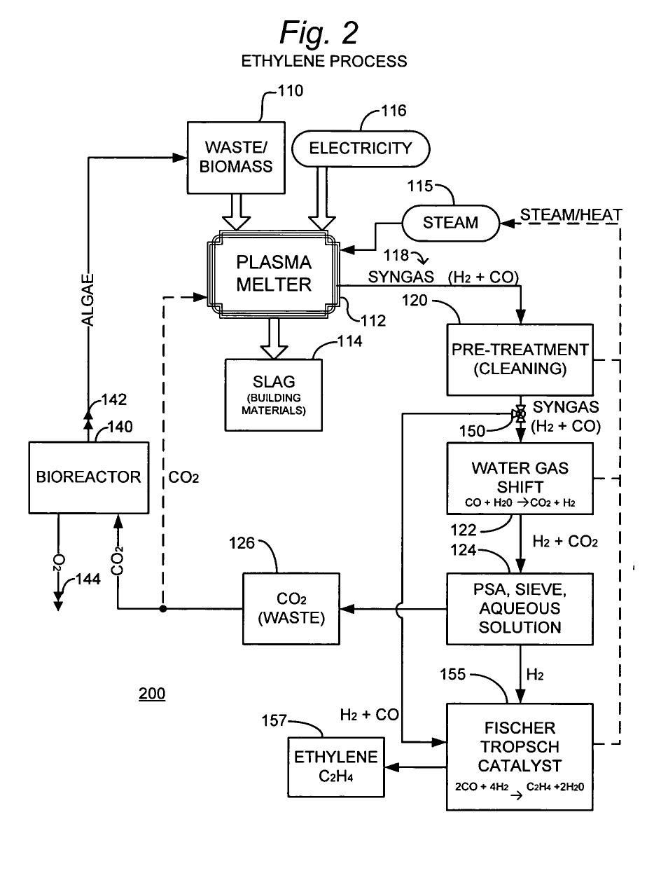

FIG. 2 is a simplified schematic representation of an embodiment of the invention wherein ethylene product is produced along with algae that is used as a fuel. Elements of structure that have previously been discussed are similarly designated.

In this specific illustrative embodiment of the invention, a portion of the CO and hydrogen obtained from pretreatment step 120 is diverted by a flow control valve 150 and supplied to a Fischer Tropsch Catalyst process 155. In some embodiments of the invention, the Fischer Tropsch Catalyst process is an iron-based Fischer Tropsch Catalyst process. This diverted flow is applied to achieve an appropriate molar ratio of CO and hydrogen, and thereby optimize the production of ethylene 157 or other carbon-based products.

Pretreatment step 120, Water Gas Shift reaction 122, and Fischer Tropsch Catalyst process 155 generate heat that in some embodiments of the invention is used to supply steam to the plasma melter 112, or to a turbine generator (not shown), or any other process (not shown) that utilizes heat.

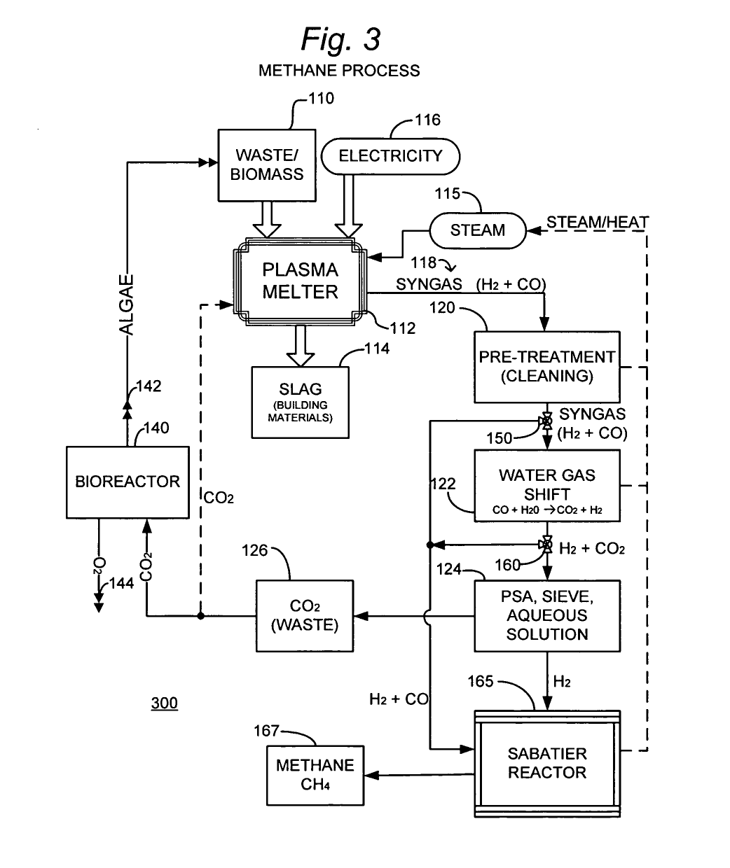

FIG. 3 is a simplified schematic representation of an embodiment of the invention 300 wherein methane product is produced along with algae that is used as a fuel. Elements of structure that have previously been discussed are similarly designated.

In this specific illustrative embodiment of the invention, a portion of the carbon monoxide and hydrogen obtained from pretreatment step 120 is diverted by a flow control valve 150 and supplied to Sabatier Reactor 165. This diverted flow is applied to achieve an appropriate molar ratio of carbon monoxide and hydrogen, and thereby optimize the production of methane. In addition, in this specific illustrative embodiment of the invention, a flow valve 160 diverts a portion of the hydrogen and carbon dioxide that is produced at the output of Water Gas Shift reaction 122 to Sabatier Reactor 165.

Pretreatment step 120, Water Gas Shift reaction 122, and Sabatier Reactor 165 generate heat that in some embodiments of the invention is used to supply steam to the plasma melter 112, or to a turbine generator (not shown), or any other process (not shown) that utilizes heat.

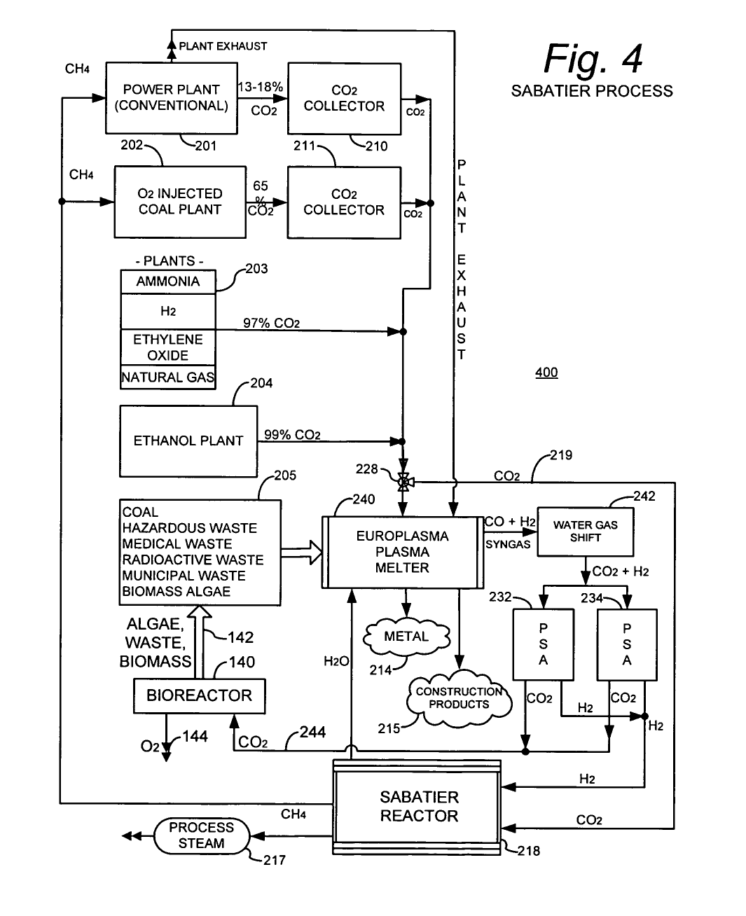

FIG. 4 is a simplified schematic representation of a still further specific illustrative embodiment of the invention, utilizing a Europlasma plasma melter and wherein methane product is produced along with algae that is used as a fuel. Elements of structure that have previously been discussed are similarly designated. In addition, other embodiments can, in light of this teaching, be produced by persons of skill in the art using other forms of plasma melters, such as an InEnTec plasma enhanced melter, or a Westinghouse plasma melter.

As shown in this figure, a carbon dioxide recycling system 400 includes a power plant 201, which in this embodiment of the invention is a conventional coal power plant having a base load, in this specific illustrative embodiment of the invention, of 1830 MW per day. In some embodiments of the invention, however, power plant 201 is powered by natural gas. In embodiments where power plant 201 is a modern coal plant, it will emit on average about 3,458,700 Lbs of carbon dioxide per hour, or about 13 to 18% of its exhaust stream by volume.

Carbon dioxide recycling system 400 additionally is provided with an oxygen enriched coal power plant 202. Oxygen enriched coal power plant 202 issues a higher concentration of carbon dioxide in its exhaust stream, i.e., about 65% by volume. Other industrial plants 203 and 204 are also included in carbon dioxide recycling system 200. Industrial plant 203, for example, includes in this specific illustrative embodiment of the invention an ammonia plant, an H₂ plant, an ethylene oxide plant, and a natural gas plant. These plants issue a carbon dioxide output concentration of approximately 97% by volume. Ethanol plant 204 is, in some embodiments, a modern plant that issues approximately 99% carbon dioxide by volume.

Carbon dioxide collectors 210 and 211 (or flue gas reactors) are carbon dioxide sequestering systems. Such systems are commercially available from suppliers such as Alstrom. In this embodiment, carbon dioxide collector 210 receives the carbon dioxide output of power plant 201, and carbon dioxide collector 211 receives the carbon dioxide output of oxygen enriched coal power plant 202. The carbon dioxide outputs of carbon dioxide collector 210, carbon dioxide collector 211, plants 203, and ethanol plant 204, are combined, in this embodiment of the invention, as carbon dioxide 219 and delivered to a Sabatier reactor 218.

A water gas shift reactor 242 is included in this specific illustrative embodiment of the invention for applications that require maximum hydrogen yield to optimize the methane conversion in Sabatier reactor 218. This will further reduce the greenhouse gas carbon dioxide by increasing the processing capability of the Sabatier reactor. Carbon dioxide waste stack 244 emits “carbon neutral” carbon dioxide since the carbon dioxide will, in some embodiments, be reclaimed from waste.

In a highly advantageous embodiment of the present invention, a plasma enhanced melter 240, which may be of the type known as a Europlasma Plasma Melter, is used generate, inter alia, syngas comprised of CO and H₂. Conventional electrolysis can be used in some embodiments to generate hydrogen, but the feed stock of municipal waste 205 with its paid tipping fee and its liberation of significant energy and reclaimed useful materials make the use of a plasma enhanced melter the preferred choice.

Europlasma Plasma Melter 240 generates a net positive outflow of usable energy (ignoring the stored energy in municipal waste) and produces no additional pollution, or carbon footprint. The primary desired output of plasma enhanced melter 240 is hydrogen rich synthesis gas (syngas) that is piped to Sabatier reactor 218. As shown in this figure, the hydrogen rich synthesis gas is delivered in parallel with carbon dioxide 219 to Sabatier reactor 218.

In one implementation of the invention, Sabatier reactor 218 is a ceramic foam Sabatier reactor. However, other forms of fuel producing endothermic reactors can be used in the practice of the invention. The close coupling of a sympathetic endothermic reaction is not required, but renders the process more energy efficient. The Sabatier reactor operates to effect the following reaction:

CO₂+4H₂═CH₄+2H₂O

The primary desired output of carbon dioxide recycling system 400 is methane (CH₄) at the output of Sabatier reactor 218, which is reburned, in this specific illustrative embodiment of the invention, in power plant 201 and oxygen enriched coal power plant 202. Reclaimed metals 214 and silica based construction materials 215 are additional benefits of plasma enhanced melter 220.

In essence, the carbon dioxide that is emitted by power plant 201 and oxygen enriched coal power plant 202 is continuously recycled, bringing its carbon foot print closer to zero and vastly increasing the efficiency of such plants, thereby reducing the amount of coal required per kilowatt-hour of power produced. However, the use of bioreactor 140 in this embodiment can reduce the carbon foot print to less than zero

In some embodiments of the invention, Sabatier reactor 218 is jacketed (not shown) in a steam generating heat transfer system (not specifically designated). Such jacketing is particularly advantageous when combined with the alumina ceramic design of the Sabatier reactor in this embodiment of the invention. The combination of the superior heat transfer of the alumina ceramic material with a steam generator increases the heat recovery efficiency of the system. Steam 217, as well as stored energy recovered from Sabatier reactor 218 is in this embodiment of the invention, returned to power plant 201 and oxygen enriched coal power plant 202, or it can be sold locally to surrounding industries (not shown), or as municipal steam for heating.

In this embodiment of the invention, there are provided pressure swing absorbers 232 and 234 (PSAs) that serve to separate the hydrogen from the CO₂. A number of other methods such as molecular sieves, and the like can be used in the practice of the invention.

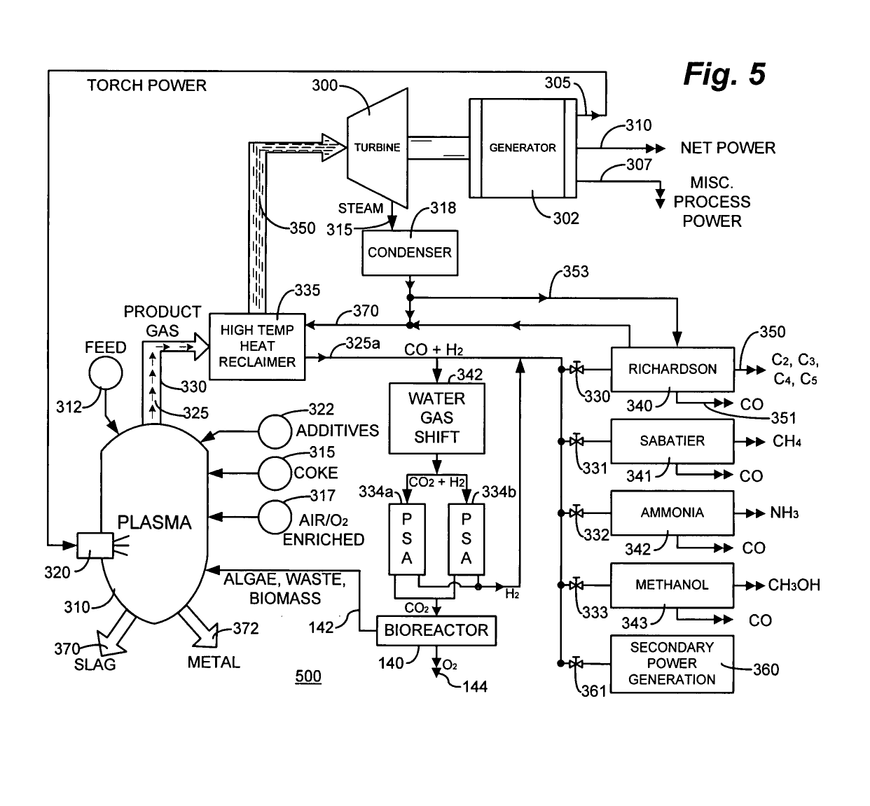

FIG. 5 is a simplified schematic representation of yet another embodiment of the invention showing a primary plant system 500 wherein algae is produced that is used as a fuel. As shown in this figure, a plasma reactor 310 will process a feedstock 312 that in this specific illustrative embodiment of the invention can consist of 100% coal, 100% municipal solid waste (MSW), 100% biomass, or any combination thereof. Other heat sources other than plasma could be used in the practice of the invention. In this embodiment, feedstock coke 315 may optionally be used. Feedstock air, or oxygen enriched air 117 also optionally may be delivered to plasma reactor 110.

Direct or indirect acting plasma torches 320 are used in this specific illustrative embodiment of the invention to excite plasma reactor 310. In a preferred mode of operation plasma reactor 310 is operated in a pyrolysis mode with compressed MSW as the feedstock. However, plasma reactor 310 can be operated in a non pyrolysis mode in the practice of the invention. Additives 322 are optionally delivered to plasma reactor 310 to neutralize the acid or base content (not specifically designated) of a product gas 325 that is conducted along an outlet duct 330. Product gas 325 exits the plasma reactor at approximately 1250° C., and approximately 27% of the total energy that is present in product gas 325 from the plasma reactor 310 primarily is in the form of sensible heat. Due to the extreme temperature and composition of product gas 325, most of the heat energy has heretofore usually been wasted. The present invention includes within its scope several methods of utilizing this energy more effectively. In this embodiment, the heat contained in product gas 325 is recovered in a high temperature heat reclamation system 335

It is shown in FIG. 5 that heated/super critical steam 350 is piped to a steam turbine 300. Steam turbine 300 is coupled to rotate a generator 302 to produce electrical energy at an electrical output 305 that is used to operate plasma torches 320. A further electrical output 307 issues electrical energy that is used to operate miscellaneous process systems (not specifically designated), and a net carbon free electrical output 310 from generator 302 constitutes net power to the distribution grid (not shown).

In a 2,500 Ton per Day (TPD) MSW plant the net continuous carbon free electrical output from this stage would be approximately 31 MW. Spent steam 315 is returned through a condenser 318 and a conduit 370, and is recharged through high temperature heat reclamation system 335, as previously described. In this specific illustrative embodiment of the invention, the spent steam that is conducted through conduit 370 includes steam obtained from a Richardson reactor 340.

It is noteworthy that the generated electrical power is actually carbon negative in this application since the typical make up of MSW contains significant amounts of biomass that captures CO₂ from the atmosphere prior to being processed in the plasma reactor 310. No additional greenhouse gas credits are produced due to the avoidance of escaping gaseous pollution from landfills. Pure biomass will produce greater power with reduced greenhouse gas emissions.

At the other extreme of the feedstock 312 scale is coal with an illustrative BTU content of approximately 14,120 btu/lb. If coal is used as feedstock 312 in a 2,500 TPD plant, the net electrical output 310 of this stage will be approximately 90 MW. This power is carbon free since no exhaust gas is released to the atmosphere in the production of this power. A combination of biomass, MSW, and coal will produce a proportionate amount of net electrical energy 310.

Product gas 325a that has been passed through high temperature heat reclamation system 335 is routed, in this specific illustrative embodiment of the invention, through control valves 330-333 to produce various products. It is to be noted that plant system 500 can employ one or more, in any combination, of reactors 340-343. In addition, some embodiments of the invention are provided with a secondary power generation system 360, wherein the CO and H₂ that are passed thought control valve 361 is compressed and provided to a secondary gas turbine (not shown) that drives a secondary generator (not shown). In still further embodiments of the secondary power generation system, heat is extracted from the exhaust of the secondary gas turbine and is used to drive yet another turbine (not shown) and further generator (not shown).

Product gas 325a that is issued by high temperature heat reclamation system 335 is routed, in this specific illustrative embodiment of the invention, through a Richardson reactor 340, which in some embodiments is a Fischer Tropsch style reactor. During off-peak electrical generation hours (e.g., at night), a base amount of carbon free, or carbon negative electrical energy is sent to the grid through generator 302. The product gas is directed to make selectively C₂, C₃, C₄, and C₅ products 350 such as plastic feed stocks through Richardson reactor 340. A small amount of CO product gas 351 is collected and sold for industrial use or product feed stock, such as detergents and polycarbonates. The CO product gas 351 is, in some embodiments of the invention, gas shifted, such as in a water gas shift process 342, to produce more hydrogen and more products 350 with a slight release of carbon neutral CO₂ or carbon positive CO₂, depending on which feed stock 312 is being used.

Product gas 325a is additionally directed to water gas shift process 342, and the shifted CO₂ and H₂ are delivered in this specific illustrative embodiment of the invention to pressure swing adsorption processes (PSAs) 334a and 334b. The CO₂ separated by the PSAs is provided to bioreactor 140 for enhancing the growth of algae 142, as noted above, as well as O₂ at outlet 144.

Each of reactors 340-343 reclaim any heat possible using steam loops, such as that designated as steam loop 353. The additional steam loops to the balance of the reactors are not shown for sake of clarity of the figure. A Sabatier Reactor 341 produces CH₄ as its output product. An ammonia process 342 produces feed stock for fertilizer or munitions, and a methanol reactor 343 produces methanol as its output product, specifically CH₃OH. In this specific illustrative embodiment of the invention, during peak electrical demand hours reactors 340-343 are bypassed by the closure of control valves 330-333, and product gas 325a is directed to secondary power generation system 360 via a control valve 361. Also, each of reactors 340-343 is shown to issue some CO, which in some embodiments of the invention, is delivered to water gas shift process 342 (conduits not shown).

Although the invention has been described in terms of specific embodiments and applications, persons skilled in the art can, in light of this teaching, generate additional embodiments without exceeding the scope or departing from the spirit of the invention herein claimed. Accordingly, it is to be understood that the drawing and description in this disclosure are proffered to facilitate comprehension of the invention, and should not be construed to limit the scope thereof.

What is claimed is:

1. A method of manufacturing ammonia on a large scale, the method comprising the steps of:
supplying a fuel material to a plasma melter;
supplying electrical energy to the plasma melter;
supplying steam to the plasma melter;
extracting a syngas from the plasma melter;
extracting H₂ and CO₂ from the syngas;
supplying at least a portion of the CO₂ extracted from the syngas to a bioreactor for enhancing the growth of algae; and
forming ammonia from the hydrogen produced in said step of extracting hydrogen;
wherein the algae forms at least a portion of the fuel material in said step of supplying the fuel materia to the plasma melter.

2. The method of claim 1, wherein said step of supplying the fuel material to the plasma melter comprises the step of supplying a selectable combination of a biomass material and municipal waste to the plasma melter.

3. The method of claim 1, wherein said step of supplying the fuel material to the plasma melter comprises the step of supplying municipal solid waste to the plasma melter.

4. The method of claim 1, wherein said step of supplying the fuel material to the plasma melter comprises the step of supplying a biomass material to the plasma melter.

5. The method of claim 4, wherein the biomass material is specifically grown for being supplied to the plasma melter.

6. The method of claim 1, wherein said step of extracting hydrogen from the syngas comprises the steps of:
subjecting the syngas to a water gas shift process to form a mixture of hydrogen and carbon dioxide; and
extracting hydrogen from the mixture of hydrogen and carbon dioxide.

7. The method of claim 6, wherein said step of extracting hydrogen from the mixture of hydrogen and carbon dioxide comprises the step of subjecting the mixture of hydrogen and carbon dioxide mixture to a pressure swing adsorption process.

8. The method of claim 6, wherein said step of extracting hydrogen from the mixture of hydrogen and carbon dioxide comprises the step of subjecting the mixture of hydrogen and carbon dioxide mixture to a molecular sieve.

9. The method of claim 6, wherein said step of extracting hydrogen from the mixture of hydrogen and carbon dioxide comprises the step of subjecting the mixture of hydrogen and carbon dioxide mixture to an aqueous ethanolamine solution.

10. The method of claim 6, wherein prior to performing said step of subjecting the syngas to a water gas shift process to form a mixture of hydrogen and carbon dioxide there is provided the step of pre-treating the output of the plasma melter to perform a cleaning of the syngas.

11. The method of claim 6, wherein prior to performing said step of subjecting the syngas to a water gas shift process to form a mixture of hydrogen and carbon dioxide there is provided the step of pre-treating the output of the plasma melter to perform a segregation of the syngas.

12. The method of claim 1, wherein said step of forming ammonia from the hydrogen produced in said step of extracting hydrogen comprises the step of subjecting the hydrogen to a Haber-Bosch process.

13. The method of claim 12, wherein prior to performing said step of forming ammonia from the hydrogen produced in said step of extracting hydrogen there is provided the further step of supplying nitrogen to the Haber-Bosch process.

14. The method of claim 13, wherein said step of supplying nitrogen to the Haber-Bosch process comprises the step of extracting nitrogen from air.

15. A method of manufacturing ethylene on a large scale, the method comprising the steps of:
supplying a fuel material to a plasma melter;
supplying electrical energy to the plasma melter;
supplying water to the plasma melter;
extracting a syngas from the plasma melter;
extracting H₂ and CO₂ from the syngas;
supplying at least a portion of the CO₂ extracted from the syngas to a bioreactor for enhancing the growth of algae; and
forming ethylene from the hydrogen produced in said step of extracting hydrogen;
wherein the algae forms at least a portion of the fuel material in said step of supplying the fuel materia to the plasma melter.

16. The method of claim 15, wherein said step of supplying water to the plasma melter comprises the step of supplying steam to the plasma melter.

17. The method of claim 15, wherein said step of supplying the fuel material to the plasma melter comprises the step of supplying a selectable combination of a biomass material and municipal waste to the plasma melter.

18. The method of claim 15, wherein said step of supplying the fuel material to the plasma melter comprises the step of supplying municipal solid waste to the plasma melter.

19. The method of claim 15, wherein said step of supplying the fuel material to the plasma melter comprises the step of supplying a biomass material to the plasma melter.

20. The method of claim 19, wherein the biomass material is specifically grown for being supplied to a plasma melter.

21. The method of claim 15, wherein said step of extracting hydrogen from the syngas comprises the steps of:
subjecting the syngas to a water gas shift process to form a mixture of hydrogen and carbon dioxide; and
extracting hydrogen from the mixture of hydrogen and carbon dioxide.

22. The method of claim 21, wherein said step of extracting hydrogen from the mixture of hydrogen and carbon dioxide comprises the step of subjecting the mixture of hydrogen and carbon dioxide mixture to a pressure swing adsorption process.

23. The method of claim 21, wherein said step of extracting hydrogen from the mixture of hydrogen and carbon dioxide comprises the step of subjecting the mixture of hydrogen and carbon dioxide mixture to a molecular sieve.

24. The method of claim 21, wherein said step of extracting hydrogen from the mixture of hydrogen and carbon dioxide comprises the step of subjecting the mixture of hydrogen and carbon dioxide to an aqueous ethanolamine solution.

25. The method of claim 21, wherein prior to performing said step of subjecting the syngas to a water gas shift process to form a mixture of hydrogen and carbon dioxide there is provided the step of pre-treating the output of the plasma melter to perform a cleaning of the syngas.

26. The method of claim 21, wherein prior to performing said step of subjecting the syngas to a water gas shift process to form a mixture of hydrogen and carbon dioxide there is provided the step of pre-treating the output of the plasma melter to perform a separation of the syngas.

27. The method of claim 15, wherein said step of forming ethylene from the hydrogen produced in said step of extracting hydrogen comprises the step of subjecting the hydrogen to a Fischer Tropsch catalytic process.

28. The method of claim 27, wherein the Fischer Tropsch Catalyst process is an iron-based Fischer Tropsch Catalyst process.

29. The method of claim 27, wherein prior to performing said step of forming ethylene from the hydrogen produced in said step of extracting hydrogen there is provided the further step of optimizing the production of ethylene by correcting the molar ratio of carbon monoxide and hydrogen in the Fischer Tropsch catalytic process.

30. The method of claim 29, wherein said step of correcting the molar ratio of carbon monoxide and hydrogen in the Fischer Tropsch catalytic process comprises the step of supplying a mixture of hydrogen and carbon monoxide to the Fischer Tropsch catalytic process.

31. The method of claim 30, wherein said step of supplying the mixture of hydrogen and carbon monoxide to the Fischer Tropsch process comprises the step of diverting a portion of the hydrogen and carbon monoxide produced by the plasma melter.

32. The method of claim 31, wherein said step of diverting a portion of the hydrogen and carbon monoxide produced by the plasma melter is performed after performing a step of cleaning the hydrogen and carbon monoxide produced by the plasma melter.

33. A method of manufacturing methane on a large scale, the method comprising the steps of:
supplying a fuel material to a plasma melter;
supplying electrical energy to the plasma melter;
supplying steam to the plasma melter;
extracting a syngas from the plasma melter;
extracting H₂ and CO₂ from the syngas;
supplying at least a portion of the CO₂ extracted from the syngas to a bioreactor for enhancing the growth of algae; and
forming methane from the hydrogen produced in said step of extracting hydrogen;
wherein the algae forms at least a portion of the fuel material in said step of supplying the fuel materia to the plasma melter.

34. The method of claim 33, wherein said step of extracting hydrogen from the syngas comprises the steps of:
subjecting the syngas to a water gas shift process to form a mixture of hydrogen and carbon dioxide; and
extracting hydrogen from the mixture of hydrogen and carbon dioxide.

35. The method of claim 34, wherein said step of extracting hydrogen from the mixture of hydrogen and carbon dioxide comprises the step of subjecting the mixture of hydrogen and carbon dioxide mixture to a pressure swing adsorption process.

36. The method of claim 34, wherein said step of extracting hydrogen from the mixture of hydrogen and carbon dioxide comprises the step of subjecting the mixture of hydrogen and carbon dioxide mixture to a molecular sieve.

37. The method of claim 15, wherein said step of extracting hydrogen from the mixture of hydrogen and carbon dioxide comprises the step of subjecting the mixture of hydrogen and carbon dioxide mixture to an aqueous ethanolamine solution.

38. The method of claim 15, wherein prior to performing said step of subjecting the syngas to a water gas shift process to form a mixture of hydrogen and carbon dioxide there is provided the step of pre-treating the output of the plasma melter to perform a cleaning of the syngas.

39. The method of claim 33, wherein said step of forming methane from the hydrogen produced in said step of extracting hydrogen comprises the step of subjecting the hydrogen to a Sabatier Reactor process.

40. The method of claim 39, wherein the step of subjecting the hydrogen to a Sabatier Reactor process is performed in a Sabatier Reactor formed of a ceramic foam material.

41. The method of claim 40, wherein the ceramic foam material has an additive incorporated therein.

42. The method of claim 41, wherein the ceramic foam material is an aluminum based ceramic foam material.

43. The method of claim 39, wherein prior to performing said step of forming methane from the hydrogen produced in said step of extracting hydrogen there is provided the further step of optimizing the production of methane by correcting the molar ratio of carbon monoxide and hydrogen in the Sabatier Reactor process.

44. The method of claim 43, wherein said step of correcting the molar ratio of carbon monoxide and hydrogen in the Sabatier Reactor process comprises the step of supplying a mixture of hydrogen and carbon monoxide to the Sabatier Reactor process.

45. The method of claim 44, wherein said step of supplying the mixture of hydrogen and carbon monoxide to the Sabatier Reactor process comprises the step of diverting a portion of the hydrogen and carbon monoxide produced by the plasma melter.

46. The method of claim 45, wherein said step of diverting a portion of the hydrogen and carbon monoxide produced by the plasma melter is performed after performing a step of cleaning the hydrogen and carbon monoxide produced by the plasma melter.

47. The method of claim 39, wherein prior to performing said step of forming methane from the hydrogen produced in said step of extracting hydrogen there is provided the further step of optimizing the production of methane by correcting the molar ratio of carbon dioxide and hydrogen in the Sabatier Reactor process.

48. The method of claim 47, wherein said step of supplying the mixture of hydrogen and carbon dioxide to the Sabatier Reactor process comprises the step of diverting a portion of the hydrogen and carbon dioxide produced by a water gas shift process.

49. The method of claim 33, wherein there is further provided the step of extracting a slag from the plasma melter.

50. The method of claim 33, wherein said step of supplying a biomass material to the plasma melter comprises the step of supplying municipal waste to the plasma melter.

51. The method of claim 33, wherein the plasma melter is operated in a pyrolysis mode.

52. A method of reclaiming carbon dioxide in an industrial process, the method comprising the steps of:
obtaining an output gas from the industrial process;
delivering the output gas to a plasma melter;
delivering a fuel material to the plasma melter;
extracting CO and H₂ from the plasma melter;
converting the CO and H₂ into CO₂ and H₂;
delivering at least a portion of the CO₂ and H₂ to a reactor wherein the CO₂ and H₂ are converted to CH₄ and steam;
returning the CH₄ to the industrial process; and
delivering at least a portion of the CO₂ obtained in said step of converting the CO and H₂ into CO₂ and H₂ to a bioreactor for enhancing the growth of algae.

53. The method of claim 52, wherein the output gas from the industrial process is CO₂.

54. The method of claim 53, wherein prior to performing the step of delivering the output gas to a plasma melter there is provided the step of collecting the CO₂ from the industrial process.

55. The method of claim 52, wherein the output gas from the industrial process is an exhaust gas.

56. The method of claim 52, wherein said step of converting the CO and H₂ into CO₂ and H₂ comprises the further steps of:
subjecting the CO and H₂ to a water gas shift process; and
extracting the H₂ and CO₂ in a pressure swing adsorption (PSA) process.

57. The method of claim 52, wherein said step of delivering at least a portion of the CO₂ and H₂ to a reactor wherein the CO₂ and H₂ are converted to CH₄ and steam comprises the further step of delivering at least a portion of the CO₂ and H₂ to a Sabatier Reactor.

58. The method of claim 52, wherein the industrial process is a power plant.

59. The method of claim 58, wherein the power plant issues a plant exhaust and there is provided the further step of delivering the plant exhaust to the plasma melter.

60. The method of claim 52, wherein there is provided the further step of delivering at least a portion of the algae as a fuel material to the plasma melter.

61. A system for generating electrical power, the system comprising:
a reactor for producing a product gas in response to the consumption of a feedstock;
a heat reclamation arrangement for extracting heat from the product gas and forming heated steam;
a turbine having an input for receiving the heated steam, an outlet for exhausting spent steam, and a rotatory output;
an electrical generator coupled to the rotatory output of said turbine for producing electrical energy; and
a bioreactor arranged to receive CO₂ from said heat reclamation arrangement for enhancing the growth of algae.

62. The system of claim 61, wherein there is further provided the delivery of at least a portion of the algae as a fuel material to the plasma melter

63. The system of claim 61, wherein there is further provided a recirculating system for returning the spent steam to said heat reclamation arrangement.

64. The system of claim 61, wherein said heat reclamation arrangement comprises:
a first duct having an inlet for receiving the product gas, and an outlet for exhausting the product gas at a reduced temperature;
a second duct having an inlet for receiving the spent steam and an outlet for exhausting the heated steam; and
a heat transfer arrangement for conducting heat extracted from the product gas in said first duct to the spent steam in said second duct, to form the heated steam.

65. The system of claim 64, wherein said heat transfer arrangement comprises a sodium heat tube having a first end for communicating with the product gas in said first duct, and a second end for communicating with the spent steam in said second duct.

66. The system of claim 65, wherein said sodium heat pipe comprises an envelope formed of stainless steel.

67. The system of claim 65, wherein said sodium heat pipe comprises an envelope formed of a selectable combination of Inconel, molybdenum, tungsten, niobium, carbon, carbon composite, and Hastelloy X.

68. The system of claim 65, wherein said sodium heat pipe comprises an envelope formed of molybdenum.

69. The system of claim 65, wherein said sodium heat pipe comprises an envelope formed of tungsten.

70. The system of claim 65, wherein said sodium heat pipe comprises an envelope formed of niobium.

71. The system of claim 65, wherein said sodium heat pipe comprises an envelope formed of a selectable combination of carbon and carbon composite.

72. The system of claim 65, wherein said sodium heat pipe comprises an envelope formed of Hastelloy X.

73. The system of claim 65, wherein said sodium heat pipe is provided with a safety valve for ensuring safe operation.

74. The system of claim 65, wherein there is further provided a heat transfer fin in said first duct for enhancing the transfer of heat from the product gas to said sodium heat pipe .

75. The system of claim 65, wherein there is further provided an adiabatic zone interposed between said first and second ducts.

76. The system of claim 61, wherein said heat transfer arrangement comprises a heat transfer loop having a first portion for communicating with the product gas in said first duct, and a second portion for communicating with the spent steam in said second duct.

77. The system of claim 64, wherein there is further provided a Sabatier reactor having an inlet for receiving the product gas at a reduced temperature, and an outlet for issuing CH₄.

78. The system of claim 64, wherein there is further provided an ammonia reactor having an inlet for receiving the product gas at a reduced temperature, and an outlet for issuing NH₃.

79. The system of claim 64, wherein there is further provided a methanol reactor having an inlet for receiving the product gas at a reduced temperature, and an outlet for issuing CH₃OH.

80. The system of claim 64, wherein there is further provided a secondary power generation facility having an inlet for receiving the product gas at a reduced temperature, and an outlet for issuing electrical power.

81. The system of claim 80, wherein said secondary power generation facility comprises:
a compressor having an inlet for receiving the product gas at a reduced temperature, and an outlet for issuing a syngas;
a first turbine for receiving the syngas, said first turbine having a rotatory output and an exhaust outlet; and
a first generator coupled to the rotatory output of said first turbine, said first generator having an output for issuing electrical power.

82. A method of operating an electrical power plant, the method comprising the steps of:
delivering a feedstock to a reactor to produce a product gas;
reclaiming heat from the product gas in a heat reclamation arrangement to form a super heated steam;
reclaiming CO₂ and H₂ from the product gas; and
delivering the CO₂ to a bioreactor for enhancing the growth of algae.

83. The method of claim 82, wherein there are provided the further steps of:
delivering the superheated steam to a turbine; and
rotating an electrical generator in response to said step of delivering the superheated steam to the turbine to produce electrical energy for an electrical distribution grid.

84. The method of claim 82, wherein said step of reclaiming heat from the product gas in a heat reclamation arrangement comprises the further step of transferring heat along a sodium tube between the product gas and the spent steam.

85. The method of claim 82, wherein said step of reclaiming heat from the product gas in a heat reclamation arrangement comprises the further step of circulating the spent steam through a conduit disposed in communication with the product gas.

86. The method of claim 82, wherein said step of reclaiming heat from the product gas in a heat reclamation arrangement comprises the further step of circulating a salt-based fluid through a conduit disposed in communication with the product gas and with the spent steam.

87. The method of claim 86, wherein said step of circulating a salt-based fluid comprises the further step of pumping the salt-based fluid through the conduit.

88. The method of claim 82, wherein said step of delivering the feedstock to the reactor comprises the step of delivering a selectable combination of coal, municipal solid waste, and a biomass material to the reactor.

89. The method of claim 82, wherein said step of delivering the feedstock to the reactor comprises the step of delivering a non-fossil fuel to the reactor.

90. The method of claim 82, wherein said step of delivering the feedstock to the reactor to produce a product gas is performed continuously independently of the demand for electrical power on the electrical distribution grid.

91. The method of claim 82, wherein there is provided the further step of operating the reactor in a pyrolysis mode.

92. The method of claim 82, wherein the reactor is a plasma reactor.

93. The method of claim 82, wherein there is provided the step of operating a Fischer Tropsch style reactor for making a product in response to a decreased demand for electrical power by the electrical distribution grid.

94. The method of claim 82, wherein there is provided the step of operating a Richardson reactor for making a product in response to a decreased demand for electrical power by the electrical distribution grid.

95. The method of claim 82, wherein there is provided the step of operating a Sabatier reactor for making a product in response to a decreased demand for electrical power by the electrical distribution grid.

96. The method of claim 82, wherein there is provided the step of operating an ammonia process for making a product in response to a decreased demand for electrical power by the electrical distribution grid.

97. The method of claim 82, wherein there is provided the step of operating a methanol process for making a product in response to a decreased demand for electrical power by the electrical distribution grid.

98. The method of claim 82, wherein there is provided the step of operating a methanol process for making a product in response to a decreased demand for electrical power by the electrical distribution grid.

99. The method of claim 82, wherein there is provided the step of operating a secondary electrical generation arrangement for producing additional electrical power in response to an increased demand for electrical power by the electrical distribution grid.

100. The method of claim 99, wherein said step of operating a secondary electrical generation arrangement is performed in response to the production of the product gas by the reactor.
